(19) United States
(12) Patent Application Publication
(10) Pub. No.: US 2009/0029954 A1
(43) Pub. Date: Jan. 29, 2009

(54) PROGESTERONE-RECEPTOR ANTAGONIST FOR USE IN BRCA ALONE OR AS COMBINATION WITH ANTIESTROGEN

(76) Inventors: Jens HOFFMANN, Muhlenbeck (DE); Daniel Korr, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/106,554

(22) Filed: Apr. 21, 2008

Related U.S. Application Data
(60) Provisional application No. 60/914,372, filed on Apr. 27, 2007.

(30) Foreign Application Priority Data
Apr. 23, 2007 (EP) 07090083.2

(51) Int. Cl. A61K 31/56

(52) U.S. Cl. 514/170; 514/171

(57) ABSTRACT

The present invention relates to the single use of the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof for the prophylaxis and treatment of BRCA1- or BRCA2-mediated breast cancer, as well as to a combination comprising the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof, together with at least one pure antiestrogen, for the prophylaxis and treatment of BRCA1- or BRCA2-mediated breast cancer, ovarian cancer endometrial cancer, gastric cancer, colorectal cancer, endometriosis, myeloma, myoma and meningioma.

This application claims the benefit of the filing date of U.S. Provisional Application Ser. No. 60/914,372 filed Apr. 27, 2007, which is incorporated by reference herein.

The present invention relates to the single use of the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof for the prophylaxis and treatment of BRCA1- or BRCA2-mediated breast cancer, as well as to a combination comprising the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof, together with at least one pure antiestrogen, for the prophylaxis and treatment of BRCA1- or BRCA2-mediated breast cancer, ovarian cancer endometrial cancer, gastric cancer, colorectal cancer, endometriosis, myeloma, myoma and meningioma.

The present invention also relates to a combination of the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof, together with at least one EGF or EGFR targeting drug, for the prophylaxis and treatment of BRCA1- or BRCA2-mediated breast cancer, ovarian cancer endometrial cancer, gastric cancer, colorectal cancer, endometriosis, myeloma, myoma and meningioma.

The progesterone-receptor antagonist 11β-(4-acetylphenyl)-17β-hydroxy-17α-(1,1,2,2,2-pentafluoroethyl)-estra-4,9-dien-3-one, also known as ZK230211 or ZK-PRA,

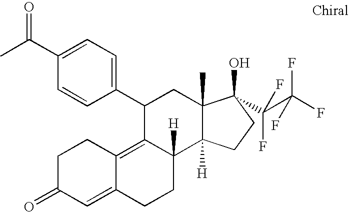

has high antiprogestagenic activity with little or no other endocrinological effects (Fuhrmann, U. et al., J. Med. Chem. 2000, 43, 5010-5016).

BRCA1 and BRCA2 are so-called tumor suppressors, genes that in their normal form protect against cancer. One way they do this is by helping cells repair DNA damage that might otherwise result in cancer-causing mutations. In Poole et al., Science, Vol. 314, 12/2006 it is described that the tumor suppressor gene BRCA-1- or BRCA2 participates in the degradation of the progesterone receptor, the gene's protein product apparently controls the progesterone growth-promoting action on breast tissue.

It is shown that mifepristone, an unspecific antiprogestin, blocks the development of mammary tumors in mice that have had the rodent version of BRCA1- or BRCA2 inactivated in their mammary glands. It is further postulated that mifepristone mediated inhibition of mammary tumorgenesis in their Brca1/p53-deficient model provides a molecular framework for future clinical evaluation of antiprogesterones as a potential chemopreventive strategy in women who carry BCRAL1- or BRCA2 mutations. However, nothing is described with respect to the activity and reaction of 11β-(4-acetylphenyl)-17β-hydroxy-17α-(1,1,2,2,2-pentafluoroethyl)-estra-4,9-dien-3-one alone or in combination with an pure antiestrogen.

Rosen et al. describe that normal BRCA1- or BRCA2 inhibits the action of the progesterone receptor, however nothing is mentioned about the mechanism. Endocrine therapy represents a mainstay of effective, minimally toxic, palliative treatment for metastatic breast cancer. As a standard palliative treatment of non-operable mammary carcinomas as well as for adjuvant therapy after primary treatment of mammary carcinomas, antiestrogens, such as the non-steroidal antiestrogen tamoxifen, are used. However, tamoxifen cannot cure breast cancer. Thus, for secondary therapy progestins or aromatase inhibitors are commonly used. In premenopausal women ovariectomy, tamoxifen and LHRH (luteinizing hormone releasing hormones) analogs achieve comparable results (H. T. Mouridson et al., Eur. J. Cancer Clin. Oncol., 24, pp. 99-105, 1988). Although tamoxifen is widely used for adjuvant therapy of breast cancer, its use as a chemopreventive agent is problematic, because it has been shown that the treatment results in an increase in the incidence of endometrial cancers (I. N. White, Carcinogenesis, 20(7):1153-60, 1999; L. Bergman et al., The Lancet, Vol. 356, Sep. 9, 2000).

Selective Progesterone-receptor antagonists (also termed as antiprogestins) represent a relatively new and promising class of therapeutic agents that could have significant impact on cancer treatment. Certain progesterone-receptor antagonists have recently gained importance in the endocrine therapy of those cancers possessing receptors for progesterone (Nathalie Chabbert-Buffet et al, Human Reproduction Update, Vol. 11, No. 3, 293-307, 2005).

This new strategy in endocrine therapy is based on the antitumor activity of progesterone-receptor antagonists in progesterone receptor-positive human breast cancer cell lines in vitro and in several hormone-dependent mammary tumors of the mouse and rat in vivo. In particular, the antitumor mechanism of the progesterone-receptor antagonists onapristone and mifepristone (RU 486) was investigated using the hormone-dependent MXT mammary tumor model of the mouse as well as the DMBA- and the MNU-induced mammary tumor models of the rat (M. R. Schneider et al., Eur. J. Cancer Clin. Oncol., Vol. 25, No. 4, pp. 691-701, 1989; H. Michna et al., Breast Cancer Research and Treatment 14:275-288, 1989; H. Michna, J Steroid. Biochem. Vol. 34, Nos 1-6, pp. 447-453, 1989). However, due to low activity and adverse side effects involved with e.g. mifepristone these compounds could not be recommended as a single agent in the management of breast cancer (D. Perrault et al., J. Clin. Oncol. 1996 October, 14(10), pp. 2709-2712).

RU 486 is causing severe side effects because of its strong anti-glucocorticoidially activity. This prohibits long term use.

When using RU 486, a further problem is for instance the poor bioavailability when administered orally. Thus, the compound generally had to be administered in high doses, giving rise to possible unfavorable side effects. Moreover, oral administration is desirable with respect to patient convenience and compliance.

Furthermore, there is still a need for compounds, alone or in combination that are active not only in the treatment, but also in the prophylaxis of breast cancer and other hormone-dependent diseases.

It has been found that the growth of hormone-dependent tumors depend, among others, e.g. on estrogens, progesterones and even testosterones. For example, most mammary carcinomas exhibit estrogen as well as progesterone receptors. Thus, a combination of progesterone-receptor antagonists together with antiestrogens may be effective in the therapy of pre- and postmenopausal mammary carcinomas.

It is thus the object of the present invention to provide a highly efficient tool for prophylaxis and treatment of especially breast cancer development and other diseases dependent upon progesterone in BRCA1- or BRCA2 mutations bearing women, such as ovarian cancer, endometrial cancer, colorectal cancer, gastric cancer, endometriosis, myeloma, myoma and meningioma.

It has now surprisingly been found that 11β-(4-acetylphenyl)-17β-hydroxy-17α-(1,1,2,2,2-pentafluoroethyl)-estra-4,9-dien-3-one alone, or as combination together with at least one pure antiestrogen can be used for the prophylaxis and treatment of BRCA1- or BRCA2-mediated breast cancer, ovarian cancer, endometrial cancer, and colorectal cancer.

It has now further most surprisingly been found that the combination of the progesterone-receptor antagonist 11β-(4-acetylphenyl)-17β-hydroxy-17α-(1,1,2,2,2-pentafluoroethyl)-estra-4,9-dien-3-one together with an antiestrogen shows a synergistic effect when compared to the inhibition of the progesterone-receptor antagonist and pure antiestrogens alone.

Antiestrogenes which can be combined together with the compound 11β-(4-acetylphenyl)-17β-hydroxy-17α-(1,1,2,2,2-pentafluoroethyl)-estra-4,9-dien-3-one are for example those which have been disclosed in WO03/045972 and which have the general formula I

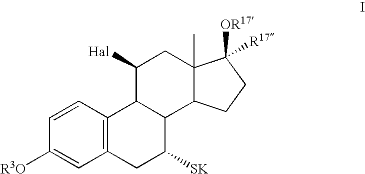

in which
Hal stands for F or Cl, and is bonded to the estratriene skeleton in 11β-position,
R³ stands for hydrogen, C₁-C₄-alkyl, C₁-C₄-alkanoyl or a cyclic C₃-C₇-ether with an O atom,
R^{17′} stands for hydrogen, C₁-C₄-alkyl or C₁-C₄-alkanoyl,
R^{17″} stands for C₁-C₄-alkyl, C₁-C₄-alkyl, C₁-C₄-alkinyl as well as for at least partially fluorinated C₁-C₄-alkyl radicals, whereby R^{17′}—O in 17β-position and R^{17″} in 17α-position are bonded to the estratriene skeleton, and
SK stands for the grouping U-V-W-X-Y-Z-E, whereby this grouping is bonded to the estratriene skeleton via U in 7α-position,
   in which U represents either a straight-chain or branched-chain C₁-C₁₃-alkylene-, -alkenylene- or -alkinylene radical or the group A-B, whereby A is bonded to the estratriene skeleton and represents a benzylidene radical that is bonded via —CH₂— to the estratriene skeleton, a phenylene radical, or a C₁-C₃-alkylaryl radical that is bonded via the alkyl group to the estratriene skeleton, and B stands for a straight-chain or branched-chain C₁-C₁₃-alkylene-, -alkenylene- or -alkinylene radical, and whereby A and B can also be connected to one another via an O atom,
   in which V further represents a CH₂— or a C(O) group,
   in which W further is an N(R⁶)— group or an N⁺(O⁻)(R⁶) group or an azolidinylene ring or an azolidinylene-N-oxide ring, whereby the azolidinylene ring or azolidinylene-N-oxide ring includes at least one C atom of grouping X, whereby R⁶ further is either H or CH₂—R⁷ or C(O)—R⁷, in which R⁷ can mean the following:
   a) hydrogen or
   b) a straight-chain or branched-chain, non-fluorinated or at least partially fluorinated C₁-C₁₄-alkyl-, -alkenyl- or -alkinyl radical, which can be hydroxylated in one or more places and can be interrupted by one to three of the heteroatoms —O— and —S— and/or the groupings —NR⁹—, in which R⁹ stands for hydrogen or a C₁-C₃-alkyl radical, or
   c) an unsubstituted or substituted aryl- or heteroaryl radical or
   d) an unsubstituted or substituted C₃-C₁₀-cycloalkyl radical or
   e) an unsubstituted or substituted C₄-C₁₅-cycloalkylalkyl radical or
   f) an unsubstituted or substituted C₇-C₂₀-aralkyl radical or
   g) an unsubstituted or substituted heteroaryl-C₁-C₆-alkyl radical or
   h) an unsubstituted or substituted aminoalkyl radical or a biphenyl radical, in which X further is a straight-chain or branched-chain C₁-C₁₂-alkylene-, -alkenylene- or -alkinylene radical,
   in which Y further is a direct bond between X and Z or can mean the following:
   a) an SOₙ—R¹⁰ group, whereby n=0, 1 or 2, only if W is an N⁺(O⁻)(R⁶) group or an azolidinylene-N-oxide ring and not an N(R⁶) group or an azolidinylene ring,
   whereby R¹⁰ represents a direct bond between SOₙ and Z or a straight-chain or branched-chain C₁-C₆-alkylene-, -alkenylene- or -alkinylene radical, or
   b) the group R¹¹ or O—R¹¹, whereby R¹¹ stands for
      i) a straight-chain or branched-chain C₁-C₅-alkylene-, -alkenylene- or -alkinylene radical or for
      ii) an unsubstituted or substituted aryl radical or heteroaryl radical or for
      iii) an unsubstituted or substituted C₃-C₁₀-cycloalkyl radical or for
      iv) an unsubstituted or substituted C₄-C₁₅-cycloalkylalkyl radical or for
      v) an unsubstituted or substituted C₇-C₂₀-aralkyl radical or for
      vi) an unsubstituted or substituted heteroaryl-C₁-C₆-alkyl radical, or
   c) the grouping CH═CF or
   d) the grouping HN—C(O)—NH—R¹², whereby R¹² stands for an unsubstituted or substituted arylene radical, and whereby R¹² is bonded to Z, and
   in which Z further is a direct bond between Y and E or a straight-chain or branched-chain C₁-C₉-alkylene-, -alkenylene- or -alkinylene radical, which can be partially or completely fluorinated, and
   in which E further is a CF₃ group or an at least partially fluorinated aryl group, whereby pharmacologically compatible acid addition salts as well as esters are also included.

Hal in particular stands for fluorine.

R³ can be hydrogen, methyl, ethyl, n-propyl, iso-propyl, n-butyl, iso-butyl and tert-butyl, a corresponding alkanoyl (acetyl, propionyl, butanoyl) or a cyclic ether. R³ in particular stands for hydrogen, CH₃, CH₃CO or C₅H₁₀O.

R^{17′} and R^{17″} are in particular methyl, ethyl, n-propyl, iso-propyl, n-butyl, iso-butyl and tert-butyl, whereby R^{17′} in addition can also be hydrogen, acetyl, propionyl and butanoyl, and whereby in this case, the corresponding isomers can be included. In addition, R¹⁷ can be ethinyl, 1-propinyl, 2-propinyl, 1-butinyl, 2-butinyl and 3-butinyl as well as trifluoromethyl, pentafluoroethyl, heptafluoropropyl and nonafluorobutyl, whereby in this case, the corresponding isomers are also included. R^{17′} is in particular hydrogen, CH₃ or CH₃CO. R^{17″} preferably stands for methyl, ethinyl and trifluoromethyl. U can be in particular a straight-chain or branched-chain alkylene radical and in particular a methylene, ethylene, propylene, butylene, pentylene, hexylene, heptylene, octylene, nonylene, decylene, undecylene, dodecylene or tridecylene radical. U preferably stands for (CH₂)ₚ, whereby p is an integer from 2 to 10. In particular, U is preferably a butylene, pentylene, hexylene or heptylene radical. U is quite especially preferably an n-butylene radical, i.e., in the formula (CH₂)ₚ for U, p=4.

In particular, V stands for CH₂. The grouping U-V thus can be n-pentylene in a quite preferred embodiment.

In particular, W stands for the amine-N-oxide N⁺(O⁻)(R⁶) or for the amine N(R⁶), whereby R⁶ is preferably hydrogen or CH₂—R⁷, in which R⁷ stands in particular for hydrogen or methyl or ethyl. R⁶ is thus preferably hydrogen or a C₁-C₃-alkyl radical, thus in particular a methyl, ethyl, n-propyl or iso-propyl radical. In an especially preferred embodiment, W represents an N⁺(O⁻)(CH₃) group (N-methylamine-N-oxide). X preferably stands for (CH₂)_{q}, whereby q=0 or an integer from 1 to 12, thus for a direct bond between W and Y or for a straight-chain or branched methylene, ethylene, propylene, butylene, pentylene, hexylene, heptylene, octylene, nonylene, decylene, undecylene or dodecylene radical. In an especially preferred embodiment, X is an ethylene, n-propylene, n-butylene, n-pentylene, n-hexylene, n-heptylene or n-octylene radical.

In particular, Y can represent a direct bond between X and Z. If this is the case, X stands for a longer alkylene chain, thus in particular, X stands for n-hexylene, n-heptylene or n-octylene. In a preferred embodiment, Y can also be an SOₙ group, whereby n=0, 1 or 2, thus a sulfanyl group, a sulfinyl group or a sulfonyl group. If Y is an SOₙ group, X represents a rather shorter alkylene chain, in particular an n-propyl chain.

Z is preferably a direct bond between Y and E or a straight-chain or branched-chain C₁-C₇-alkylene radical, which can be at least partially fluorinated. In particular, Z can be a methylene, ethylene, propylene or butylene radical, which can be at least partially fluorinated. In particular, Z is difluoromethylene or a straight-chain alkylene radical, which is perfluorinated on one end, thus, for example, a 1,1-difluoroethylene, 1,1,2,2-tetrafluoro-n-propylene or 1,1,2,2,3,3-hexafluoro-n-butylene radical. Alkylene radicals that carry only two fluorine atoms on a terminal C-atom are especially advantageous, whereby this CF₂ group is bonded to radical E. In this case, side chain SK is terminated with C₂F₅.

In particular, E stands for CF₃ or for pentafluorophenyl. The grouping Z-E thus preferably represents one of the groups that is selected from the group that comprises C₂F₅, C₃F₇ and C₄F₉ as well as C₆F₅.

According to this invention, pharmacologically compatible acid addition salts as well as esters of 17α-alkyl-17β-oxy-estratrienes are also included. The addition salts are the corresponding salts with inorganic and organic acids. As addition salts, in particular the hydrochlorides, hydrobromides, acetates, citrates, oxalates, tartrates and methanesulfonates are considered. If R³ and R¹⁷ are hydrogen, such that a 3,17β-diol is present, the esters of these hydroxy compounds can also be formed. These esters are preferably formed with organic acids, whereby the same acids as for forming the addition salts are suitable, namely in particular acetic acid, but also higher carboxylic acids, such as, e.g., propionic, butyric, isobutyric, valeric, isovaleric or pivalic acid.

Selected compounds which can be combined together with 11β-(4-acetylphenyl)-17β-hydroxy-17α-(1,1,2,2,2-pentafluoroethyl)-estra-4,9-dien-3-one, are the following compounds:
11β-Fluoro-7α-{5-[methyl(7,7,8,8,9,9,9-heptafluorononyl)amino]pentyl}-17α-methylestra-1,3,5(10)-triene-3,17β-diol N-oxide
11β-Fluoro-7α-{5-[methyl(8,8,9,9,10,10,10-heptafluorodecyl)amino]pentyl}-17α-methylestra-1,3,5(10)-triene-3,17β-diol N-oxide
(RS)-11β-Fluoro-7α-{5-[methyl(7,7,8,8,9,9,10,10,10-nonafluorodecyl)amino]-pentyl}-17α-methylestra-1,3,5(10)-triene-3,17β-diol N-oxide
11β-Fluoro-7α-{5-[methyl(8,8,9,9,9-pentafluorononyl)amino]pentyl}-17α-methylestra-1,3,5(10)-triene-3,17β-diol N-oxide
11β-Fluoro-7α-{5-[methyl(9,9,10,10,10-pentafluorodecyl)amino]pentyl}-17α-methylestra-1,3,5(10)-triene-3,17β-diol N-oxide
11β-Fluoro-7α-{5-[methyl(8,8,9,9,9-pentafluorononyl)amino]pentyl}-17α-methylestra-1,3,5(10)-triene-3,17β-diol
11β-Fluoro-7α-{5-[methyl(7,7,8,8,9,9,10,10,10-nonafluorodecyl)amino]pentyl}-17α-methylestra-1,3,5(10)-triene-3,17β-diol
11β-Fluoro-7α-{5-[methyl(7,7,8,8,9,9,9-heptafluorononyl)amino]pentyl}-17α-methylestra-1,3,5(10)-triene-3,17β-diol
17α-Ethinyl-11β-fluoro-7α-{5-[methyl(7,7,8,8,9,9,10,10,10-nonafluorodecyl)-amino]pentyl}-estra-1,3,5(10)-triene-3,17β-diol
17α-Ethinyl-11β-fluoro-3-(2-tetrahydropyranoyloxy)-7α-{5-[methyl(7,7,8,8,9,9,10,10,10-nonafluorodecyl)amino]pentyl}-estra-1,3,5(10)-trien-17β-ol
11β-Fluoro-3-(2-tetrahydropyranyloxy)-7α-{5-[methyl(7,7,8,8,9,9,10,10,10-nonafluorodecyl)amino]pentyl}-17α-methylestra-1,3,5(10)-trien-17β-ol
11β-Fluoro-7α-{5-[methyl(7,7,8,8,9,9,10,10,10-nonafluorodecyl)amino]pentyl}-17α-trifluoromethylestra-1,3,5(10)-triene-3,17β-diol
11β-Fluoro-7α-{5-[methyl(6,6,7,7,8,8,8-heptafluorooctyl)amino]pentyl}-17α-methylestra-1,3,5(10)-triene-3,17β-diol
11β-Fluoro-7α-{5-[methyl(8,8,9,9,10,10,10-heptafluorodecyl)amino]pentyl}-17α-methylestra-1,3,5(10)-triene-3,17β-diol
11β-Fluoro-7α-{5-[methyl(6,6,7,7,8,8,9,9,10,10,10-undecafluorodecyl)amino]-pentyl}-17α-methylestra-1,3,5(10)-triene-3,17β-diol
11β-Fluoro-7α-{5-[methyl(5,5,6,6,7,7,8,8,8-nonafluorooctyl)amino]pentyl}-17α-methylestra-1,3,5(10)-triene-3,17β-diol
11β-Fluoro-7α-{5-[methyl(9,9,10,10,11,11,11-heptafluoroundecyl)amino]-pentyl}-17α-methylestra-1,3,5(10)-triene-3,17β-diol
11β-Fluoro-7α-{5-[methyl(9,9,10,10,10-pentafluorodecyl)amino]pentyl}-17α-methylestra-1,3,5(10)-triene-3,17β-diol.

These compounds show high antiestrogenic activity after administration. Due to the blockade of the 17β-position formation of (active) metabolites is suppressed.

The especially preferred antiestrogenic compounds are 11β-Fluoro-17α-methyl-7α-{5-[methyl (8,8,9,9,9-pentafluorononyl)amino]pentyl}-estra-1,3,5(10)-triene-3,17β-diol and 7 alpha-[9-(4,4,5,5,5-pentafluoropentylsulfinyl)-nonyl]-estra-1,3,5(10)-triene-3,17 beta-diol, and their pharmaceutically acceptable derivatives or analogues thereof.

Further interesting steroidal antiestrogenic compounds which can be combined with the progesterone-receptor antagonist 11β-(4-acetylphenyl)-17β-hydroxy-17α-(1,1,2,2,2-pentafluoroethyl)-estra-4,9-dien-3-one are, for example, TAS-108 (Yamamoto et al., Clin. Cancer Res. 315, Vol. 11, 315-322, 2005), which has the following structure

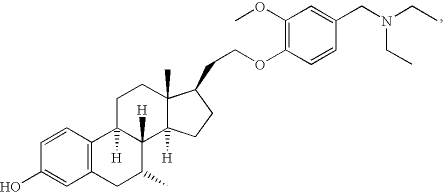

### and ICI 164.384 (Wakeling et al., Cancer Res. 51, 3867, (1991).

It has further been found that 11β-(4-acetylphenyl)-17β-hydroxy-17α-(1,1,2,2,2-pentafluoroethyl)-estra-4,9-dien-3-one, or the combination with an antiestrogen were accompanied by increased apoptosis of tumor cells, a particularly advantageous mechanism of action for the prevention or treatment of mammary carcinoma and other hormone-dependent diseases, where an indicator of high risk is an increased amount of tumor cells in the S-phase of the cell cycle. Such other hormone-dependent diseases may include ovarian cancer, endometrial cancer, myeloma, lung cancer, meningioma, i.e., diseases which substantially originate or are influenced by the presence of hormone receptors and/or hormone-dependent pathways.

The invention furthermore relates to the use of the combination for the preparation of a medicament for prophylaxis and treatment of cancer in BRCA1 and BRCA2 mutation bearing women, as well as for the treatment of other hormone-dependent conditions. In particular, 11β-(4-acetylphenyl)-17β-hydroxy-17α-(1,1,2,2,2-pentafluoroethyl)-estra-4,9-dien-3-one is particularly suitable for preventing hormone-dependent tumors, and the combination of 11β-(4-acetylphenyl)-17β-hydroxy-17α-(1,1,2,2,2-pentafluoroethyl)-estra-4,9-dien-3-one with a pure antiestrogen has been shown to effectively inhibit the growth of such tumors as compared to the progesterone-receptor antagonist or pure antiestrogen alone.

In another aspect, the present invention provides a method for prophylaxis and treatment of breast cancer and other hormone-dependent diseases in a mammal, in particular a human, in need of such treatment because of mutations in the BRCA1 or BRCA2 gene, said method comprising administering a pharmaceutically effective amount of a composition comprising the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof, and at least one pure antiestrogen to a mammal in need thereof.

11β-(4-acetylphenyl)-17β-hydroxy-17α-(1,1,2,2,2-pentafluoroethyl)-estra-4,9-dien-3-one or a pharmaceutically acceptable derivative or analogue thereof can be used according to the present invention alone or in combination with at least one pure antiestrogen. Preferably, the pure antiestrogen is selected from the group consisting of pure antiestrogens, including pharmaceutically acceptable derivatives or analogues of these pure antiestrogens. The combination is particularly advantageous for the prophylaxis and treatment of cancer and other hormone-dependent diseases.

Compounds, also known as pure antiestrogens can be used for the purposes of the present invention, for instance 7 alpha-[9-(4,4,5,5,5-pentafluoropentylsulfinyl)-nonyl]-estra-1,3,5(10)-triene-3,17 beta-diol and the pharmaceutically acceptable derivative or analogues thereof.

Although progesterone-receptor antagonist (I) is the preferred progesterone-receptor antagonist for purposes of the present invention, this does not exclude the possibility to use other suitable progesterone-receptor antagonists as well.

With regard to the superiority of the inventive combination over the prior art, it is especially favorable that the progesterone-receptor antagonist 11β-(4-acetylphenyl)-17β-hydroxy-17α-(1,1,2,2,2-pentafluoroethyl)-estra-4,9-dien-3-one shows only very weak or no endocrine side effects, such as e.g. androgen, estrogen or antiglucocorticoid activity.

Furthermore, the pure antiestrogens used according to the present invention have substantially no partial estrogen activity, compared to tamoxifen or raloxifen. The pure antiestrogens used according to the present invention, in particular the pure antiestrogen 11β-Fluoro-17α-methyl-7α-{5-[methyl(8,8,9,9,9-pentafluorononyl)-amino]pentyl}-estra-1,3,5(10)-triene-3,17β-diol exhibit a particularly high bioavailability if compared to e.g. the conventionally used antiestrogen ICI 182,780 (EP-A-0 138 504).

Due to the high bioavailability of the combination according to the present invention comprising the progesterone-receptor antagonist 11β-(4-acetylphenyl)-17β-hydroxy-17α-(1,1,2,2,2-pentafluoroethyl)-estra-4,9-dien-3-one and the pure antiestrogens, preferably pure antiestrogen 11β-Fluoro-17α-methyl-7α-{5-[methyl (8,8,9,9,9-pentafluorononyl)-amino]pentyl}-estra-1,3,5(10)-triene-3,17β-diol, including their pharmaceutically acceptable derivatives or analogues thereof, it is possible that the combination can be administered orally.

Thus, of most interest is a combination comprising the progesterone receptor antagonist 11β-(4-acetylphenyl)-17β-hydroxy-17α-(1,1,2,2,2-pentafluoroethyl)-estra-4,9-dien-3-one together with the antioestrogen 11β-Fluoro-17α-methyl-7α-{5-[methyl(8,8,9,9,9-pentafluorononyl)-amino]pentyl}-estra-1,3,5(10)-triene-3,17β-diol and their pharmaceutically acceptable derivatives and analogs thereof.

The oral administration has the advantage of improved convenience and patient compliance. As a further favorable consequence, the combination of the present invention is well tolerated. Partial agonism is commonly associated with undesirable side effects, such as for example in the case of the partial antiestrogen tamoxifen an increase in the incidence of endometrial cancers (see I. N. White, Carcinogenesis, 20(7):1153-60, 1999; L. Bergman et al., The Lancet, Vol. 356, Sep. 9, 2000, 881-887) as well as the antiglucocorticoid effects and certain toxic side effects related to the administration of the prior art progesterone-receptor antagonist mifepristone (see D. Perrault et al., J. Clin. Oncol. 1996 October, 14(10), pp. 2709-2712; L. M. Kettel et al., Fertil. Steril. 1991 September, 56(3), pp. 402-407; X. Bertagna, Psychoneuroendocrinology 1997, 22 Suppl. 1; pp. 51-55).

The pure antiestrogens if used in the amounts according to the present invention will not show the undesired side effects associated with the partial antiestrogens.

Optionally, the progesterone-receptor antagonist 11β-(4-acetylphenyl)-17β-hydroxy-17α-(1,1,2,2,2-pentafluoroethyl)-estra-4,9-dien-3-one and the pure antiestrogen, for example 11μ-Fluoro-17α-methyl-7α-{5-[methyl(8,8,9,9,9-pentafluorononyl)-amino]pentyl}-estra-1,3,5(10)-triene-3,17β-diol can further be combined with further pharmacologically active agents, such as cytotoxic agents.

The manufacture of the medicaments/pharmaceutical compositions may be performed according to methods known in the art. Commonly known and used adjuvants, as well as further suitable carriers or diluents may be used.

Suitable carriers and adjuvants may be such as recommended for pharmacy, cosmetics and related fields in: Ullmann's Encyclopedia of Technical Chemistry, Vol. 4, (1953), pp. 1-39; Journal of Pharmaceutical Sciences, Vol. 52 (1963), p. 918ff; H.v.Czetsch-Lindenwald, “Hilfsstoffe für Pharmazie und angrenzende Gebiete”; Pharm. Ind. 2, 1961, p. 72ff; Dr. H. P. Fiedler, Lexikon der Hilfsstoffe für Pharmazie, Kosmetik und angrenzende Gebiete, Cantor K G, Aulendorf in Württemberg, 1971.

The inventive combination also comprises pharmaceutical compositions, which can be prepared by known methods of preparing galenics for oral, parenteral, e.g. intraperitoneal, intramuscular, subcutaneous or percutaneous application. The inventive combination can also be implanted into tissue.

The inventive combination can also be administered in the form of tablets, pills, dragees, gel capsules, granules, suppositories, implants, injectable sterile aqueous or oily solutions, suspensions or emulsions, ointments, creams, gels, patches for transdermal administration, formulations suitable for administration by inhalation, for instance nasal sprays or by intravaginal (e.g. vaginal rings) or intrauterine systems (diaphragms, loops).

For the preparation of the pharmaceutical compositions for oral administration, the active agents suitable for the purposes of the present invention as defined above can be admixed with commonly known and used adjuvants and carriers such as for example, gum arabic, talcum, starch, sugars like e.g. mannitose, methyl cellulose, lactose, gelatin, surface-active agents, magnesium stearate, aqueous or non-aqueous excipients, paraffin derivatives, crosslinking agents, dispersants, emulsifiers, lubricants, conserving agents and flavoring agents (e.g., ethereal oils). In the pharmaceutical composition, the progesterone-receptor antagonist and the pure antiestrogen may be dispersed in a microparticle, e.g. a nanoparticulate, composition.

In order to further enhance the bioavailability of the active agents, the active agents suitable for the purposes of the present invention as defined above can also be formulated as cyclodextrin clathrates by reacting them with α-, β- or γ-cyclodextrines or derivatives thereof according to the method as disclosed in PCT/EP95/02656.

For parenteral administration the active agents suitable for the purposes of the present invention as defined above can be dissolved or suspended in a physiologically acceptable diluent, such as e.g., oils with or without solubilizers, surface-active agents, dispersants or emulsifiers. As oils for example and without limitation, olive oil, peanut oil, cottonseed oil, soybean oil, castor oil and sesame oil may be used.

The pharmaceutical compositions/medicaments according to the present invention can also be administered via a depot injection or an implant preparation, optionally for sustained delivery of the active agent(s).

Implants can comprise as inert materials e.g. biologically degradable polymers or synthetic silicones such as e.g. silicone rubber.

For percutaneous applications, the active agent(s) may also be formulated into adhesives.

The preferred mode of administration is oral administration. The combination according to the present invention, and in particular combinations comprising progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof and the pure antiestrogen 11β-Fluoro-17α-methyl-7α-{5-[methyl(8,8,9,9,9-pentafluorononyl)-amino]pentyl}-estra-1,3,5(10)-triene-3,17β-diol or pharmaceutically acceptable derivatives or analogues of both compounds, are particularly suitable for oral administration.

The inventive combination can be administered by applying the progesterone-receptor antagonist 11β-(4-acetylphenyl)-17β-hydroxy-17α-(1,1,2,2,2-pentafluoroethyl)-estra-4,9-dien-3-one together with the antiestrogens, or applying the progesterone-receptor antagonist 11β-(4-acetylphenyl)-17β-hydroxy-17α-(1,1,2,2,2-pentafluoroethyl)-estra-4,9-dien-3-one separately from the antiestrogens, for example the progesterone-receptor 11β-(4-acetylphenyl)-17β-hydroxy-17α-(1,1,2,2,2-pentafluoroethyl)-estra-4,9-dien-3-one can be administered subcutaneously or i.m. and the pure antiestrogens, for example 11β-fluoro-17α-methyl-7α-{5-[methyl(8,8,9,9,9-pentafluorononyl)-amino]pentyl}-estra-1,3,5(10)-triene-3,17β-diol can be administered orally or vice versa.

The amounts (a “pharmaceutically effective amount”) of the combined active agents to be administered vary within a broad range and depend on the condition to be treated and the mode of administration. They can cover any amount efficient for the intended treatment. Determining a “pharmaceutically effective amount” of the combined active agent is within the purview of a person skilled in the art.

The weight ratio of the progesterone-receptor antagonist 11β-(4-acetylphenyl)-17β-hydroxy-17α-(1,1,2,2,2-pentafluoroethyl)-estra-4,9-dien-3-one to the pure antiestrogen(s), as defined above, can vary within a broad range. They can either be present in equal amounts or one component can be present in excess of the other components. Preferably, 0.1 to 200 mg of the pure antiestrogen and 0.1 to 100 mg of the progesterone-receptor antagonist 11β-(4-acetylphenyl)-17β-hydroxy-17α-(1,1,2,2,2-pentafluoroethyl)-estra-4,9-dien-3-one are administered in a unit dose, more preferably in a unit dose of 10 to 150 mg of each of the pure antiestrogen and progesterone-receptor antagonist 11β-(4-acetylphenyl)-17β-hydroxy-17α-(1,1,2,2,2-pentafluoroethyl)-estra-4,9-dien-3-one. In special cases up to 200 mg of the progesterone-receptor antagonist 11β-(4-acetylphenyl)-17β-hydroxy-17α-(1,1,2,2,2-pentafluoroethyl)-estra-4,9-dien-3-one may be administered. The pure antiestrogen and progesterone-receptor antagonist 11β-(4-acetylphenyl)-17β-hydroxy-17α-(1,1,2,2,2-pentafluoroethyl)-estra-4,9-dien-3-one are preferably present in ratios from 100:1 to 1:100. More preferably, they are present in ratios from 4:1 to 1:4.

The progesterone-receptor antagonist 11β-(4-acetylphenyl)-17β-hydroxy-17α-(1,1,2,2,2-pentafluoroethyl)-estra-4,9-dien-3-one and the pure antiestrogen(s) can be administered either together or separately, at the same time and/or sequentially. Preferably they are administered combined in one unit dose. In case they are administered sequentially, preferably the progesterone-receptor antagonist 11β-(4-acetylphenyl)-17β-hydroxy-17α-(1,1,2,2,2-pentafluoroethyl)-estra-4,9-dien-3-one is administered before the pure antiestrogen(s), as defined above.

The combination of the progesterone-receptor antagonist 11β-(4-acetylphenyl)-17β-hydroxy-17α-(1,1,2,2,2-pentafluoroethyl)-estra-4,9-dien-3-one and a pure antiestrogen, for example the 11β-Fluoro-17α-methyl-7α-{5-[methyl(8,8,9,9,9-pentafluorononyl)-amino]pentyl}-estra-1,3,5(10)-triene-3,17β-diol, or pharmaceutically acceptable derivatives or analogues of these components exerts very strong tumor-inhibiting effects in a panel of hormone-dependent breast cancer models (cf. Example 1). The inhibition is synergistic when compared to the inhibition achieved by these compounds alone. The combination of progesterone-receptor antagonist 11β-(4-acetylphenyl)-17β-hydroxy-17α-(1,1,2,2,2-pentafluoroethyl)-estra-4,9-dien-3-one and a pure antiestrogen, for example the 11β-Fluoro-17α-methyl-7α-{5-[methyl(8,8,9,9,9-pentafluorononyl)-amino]pentyl}-estra-1,3,5(10)-triene-3,17β-diol is furthermore superior to a combination of progesterone-receptor antagonist 11β-(4-acetylphenyl)-17β-hydroxy-17α-(1,1,2,2,2-pentafluoroethyl)-estra-4,9-dien-3-one and the partial antiestrogen tamoxifen, which is a standard agent in breast cancer therapy (cf. Example 1).

Medicaments, such as the combination in the various aspects of the invention, that induce apoptosis in cells, for example, in the case of tumor cells, by blocking progression in the G₀G₁-phase, have potential applications for treating and preventing numerous conditions. For example, the combination of progesterone-receptor antagonist 11β-(4-acetylphenyl)-17β-hydroxy-17α-(1,1,2,2,2-pentafluoroethyl)-estra-4,9-dien-3-one and pure antiestrogen(s), may be used for treating those cancers where an indicator of high risk is an increased amount of tumor cells in the S-phase of the cell cycle, such as in breast cancer (see G. M. Clark et al., N. Engl. J. Med. 320, 1989, March, pp. 627-633; L. G. Dressler et al., Cancer 61(3), 1988, pp. 420-427 and literature cited therein).

Without limitation to any theory, the results provided in the example indicate that the main mechanism of the antitumor action of a combination of the progesterone-receptor antagonist 11β-(4-acetylphenyl)-17β-hydroxy-17α-(1,1,2,2,2-pentafluoroethyl)-estra-4,9-dien-3-one and pure antiestrogens, according to the present invention in the tested model is a direct estrogen-receptor and/or progesterone-receptor-mediated antiproliferative effect at the level of the tumor cells, via the induction of terminal differentiation associated with terminal cell death. In this manner, the combination according to the invention appears to be capable of eliminating the intrinsic block in terminal differentiation inherent in malignant tumor cells in progesterone receptor-positive and estrogen-receptor positive tumors.

Using cell cultures it was revealed that the progesterone receptor is degraded less when BRCA1- or BRCA2 activity is knocked down. As a result, the transcriptional activity of progesterone receptor by progesterone is longer and also stronger. We showed that we could reduce the accelerated PR signaling in BRCA1- or BRCA2 knocked down cells by prophylactic treatment with the instant compounds and combinations. This results in an reduced proliferation of these breast cells.

The loss in control of PR transcription may be one explanation why tumors occur specifically in the breast, ovaries and endometrium meningio organs that specifically dependent on PR, even though the BRCA1- or BRCA2 gene is mutated in cells throughout the body.

Mammary tissue of female mice bearing a similar to human BRCA1- or BRCA2 mutation (and in which p53 gene has been knocked out, showed increased cell proliferation and progesterone receptors expression and develop mammary cancers. Mice treated with the instant compounds, respectively combinations, however, were tumor free.

The effects of the instant compounds, respectively combinations may not only be restricted to tumor tissue but rather to tissue adjacent to <human> breast tumors with BRCA1- or BRCA2 mutations which also shows elevated progesterone expression compared to tissue from normal breast.

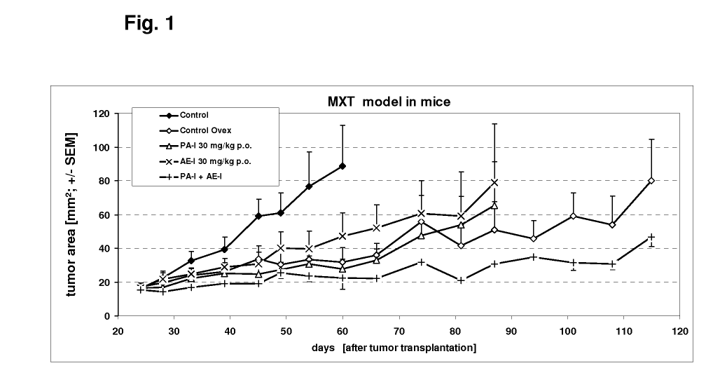

FIGS. 1/2 and 2/2 show tumor areas versus days after tumor transplantation for, antiprogestin, antiestrogen and the combination.

The invention is further illustrated in the Examples. The following Examples are, however, not to be understood as a limitation.

### EXAMPLE 1

### 11β-(4-acetyl phenyl)-17β-hydroxy-17α-(1,1,2,2,2-pentafluoroethyl)-estra-4,9-dien-3-one Inhibits Growth of Breast Cells with BRAC1 and BRCA2 Knock Down

MCF-10 mammary cells and T47D breast cancer cells obtained from ATCC were treated with siRNA knocking down the BRCA1 and BRCA2 gene.

Cell growth in comparison to untransfected and mock transfected cells was compared. In a second step cells were stimulated either with progesterone and/or with estrogens. An increased proliferation was seen in the BRCA1 and BRCA1 kno cells in the presence of progesterone. The co-treatment with 11β-(4-acetylphenyl)-17β-hydroxy-17α-(1,1,2,2,2-pentafluoroethyl)-estra-4,9-dien-3-one alone or in combination with an antiestrogen was able to antagonize the effects of BRCA1 knock down. The effects on progesteron receptor protein expression were further investigated. By using siRNA for knock down of BRCA1 an increased stability of progesterone receptor which could be antagonized by the progesterone receptor antagonist 11β-(4-acetylphenyl)-17β-hydroxy-17α-(1,1,2,2,2-pentafluoroethyl)-estra-4,9-dien-3-one was found.

Thus, the results show that the use of a progesterone-receptor antagonist, such as 11β-(4-acetylphenyl)-17β-hydroxy-17α-(1,1,2,2,2-pentafluoroethyl)-estra-4,9-dien-3-one alone or in combination with other drugs according to the present invention results in a potent inhibition of the growth of BRCA1 knock down cells.

### EXAMPLE 2

### The Roles of Progesterone Receptors in BRCA1- and BRCA2-Mediated Tumorigenesis

To address the specific roles of progesterone receptors in BRCA1- and BRCA2-mediated tumorigenesis, transgenic mice with an organ specific inactivation of p53 and BRCA1, respectively BRCA2 in the breast tissue have been studied. Inactivation of both BRCA1, respectively BRCA2 and p53 or c-myc genes in the mouse mammary gland mimics the majority of human BRCA1-associated tumors, which also harbor p53 mutations.

Because progesterone is a potent mitogen for BRCA1, respectively BRCA2 p53 or c-myc Crec mammary epithelial cells, it was tested whether blockade of the progesterone receptor activity by the progesterone receptor antagonist 11β-(4-acetylphenyl)-17β-hydroxy-17α-(1,1,2,2,2-pentafluoroethyl)-estra-4,9-dien-3-one could prevent or delay mammary carcinogenesis. Mice were treated with a placebo or with 11β-(4-acetylphenyl)-17β-hydroxy-17α-(1,1,2,2,2-pentafluoroethyl)-estra-4,9-dien-3-one. The mice were monitored weekly for tumor formation. The median tumor latency of was 6.6 months for the placebo control group with all mice developing palpable tumors. In contrast, no palpable tumors were detected in the 11β-(4-acetylphenyl)-17β-hydroxy-17α-(1,1,2,2,2-pentafluoroethyl)-estra-4,9-dien-3-one treated mice. These results suggest that the progesterone receptor function is critical for BRCA1-, respectively BRCA2-mediated mammary carcinogenesis and that treatment with PRA can prevent or delay mammary carcinogenesis.

### EXAMPLE 3

### Combination of the Progesterone Receptor Antagonist 11β-(4-acetylphenyl)-17β-hydroxy-17α-(1,1,2,2,2-pentafluoroethyl)-estra-4,9-dien-3-one Together with Faslodex Inhibits Growth of Breast Cells with BRAC1 and BRCA2 Knock Down

MCF-10 mammary cells obtained from ATCC were treated with siRNA knocking down the BRCA1 and BRCA2 gene.

Cell growth in comparison to untransfected and mock transfected cells was compared. In a second step cells were stimulated either with progesterone and/or with estrogens. An increased proliferation was seen in the BRCA1 and BRCA1 ko cells in the presence of progesterone. The co-treatment with 11β-(4-acetylphenyl)-17β-hydroxy-17α-(1,1,2,2,2-pentafluoroethyl)-estra-4,9-dien-3-one alone or in combination with an antiestrogen was able to antagonize the effects of BRCA1 knock down. The effects on progesteron receptor protein expression were further investigated. By using siRNA for knock down of BRCA1 an increased stability of progesterone receptor which could be antagonized by the progesterone receptor antagonist 11β-(4-acetylphenyl)-17β-hydroxy-17α-(1,1,2,2,2-pentafluoroethyl)-estra-4,9-dien-3-one was found.

Thus, the results show that the combination of the progesterone-receptor antagonist 11β-(4-acetylphenyl)-17β-hydroxy-17α-(1,1,2,2,2-pentafluoroethyl)-estra-4,9-dien-3-one in combination with tamoxifen or fulvestrant (Faslodex) according to the present invention results in a potent inhibition of the growth of BRCA1 knock down cells.

### EXAMPLE 4

### MXT Breast Cancer Model in Mice

### Combination of the Progesterone Receptor Antagonist 11β-(4-acetylphenyl)-171β-hydroxy-17α(1,1,2,2,2-pentafluoroethyl)-estra-4,9-dien-3-one Together with the Antiestrogen Faslodex

MXT mammary tumors obtained from donor mice are implanted in fragments of about 2 mm diameter in the inguinal region of female BDF1 mice (Charles River). Treatment is started when tumors are 25 mm² in size with
A) 1) control, 2) progesterone-receptor antagonist (I), 3) faslodex 4) combination of progesterone-receptor antagonist (I) and faslodex (I), whereby all compounds are administered daily subcutaneously or orally
B) 1) control, 2), 3) progesterone-receptor antagonist (I), 4) combination of progesterone-receptor antagonist (I) and whereby all compounds are administered daily orally

The tumor area is determined by caliper measurements. The tumor weight is determined at the end of the experiment.

Progesterone-receptor antagonist (I)=PA-I: 11β-(4-acetylphenyl)-17β-hydroxy-17α-(1,1,2,2,2-pentafluoroethyl)-estra-4,9-dien-3-one antiestrogen (Ia)=AE-I: Faslodex

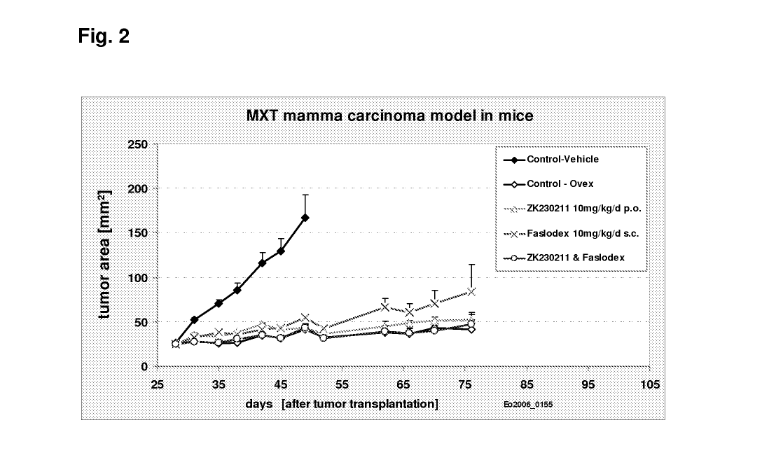

The results for the combination PRA and Faslodex are shown in FIG. 1/2 and FIG. 2/2.

Compared to the rapid growth of the control, the combination of progesterone-receptor antagonist (I) and faslodex (Ia) according to the present invention exerts an antitumor effect significantly superior to that of the single compounds. Thus, the tumor growth inhibitory effect of the composition according to the invention is an synergistic effect.

1. A pharmaceutical combination comprising the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof together with at least one pure antiestrogen for the prophylaxis and treatment of BRCA1- or BRCA2-mediated breast cancer.

2. A pharmaceutical combination according to claim 1, wherein the pure antiestrogen is selected from the group of compounds represented by general formula I 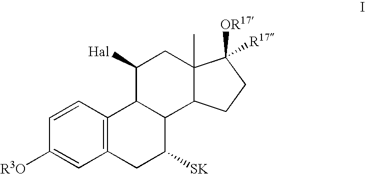
in which
Hal stands for F or Cl, and is bonded to the estratriene skeleton in 11β-position,
R³ stands for hydrogen, C₁-C₄-alkyl, C₁-C₄-alkanoyl or a cyclic C₃-C₇-ether with an O atom,
R^{17′} stands for hydrogen, C₁-C₄-alkyl or C₁-C₄-alkanoyl, R^{17″} stands for C₁-C₄-alkyl, C₁-C₄-alkyl, C₁-C₄-alkinyl as well as for at least partially fluorinated C₁-C₄-alkyl radicals,
whereby R^{17′}—O in 17β-position and R^{17″} in 17α-position are bonded to the estratriene skeleton, and
SK stands for the grouping U-V-W-X-Y-Z-E, whereby this grouping is bonded to the estratriene skeleton via U in 7α-position,
in which U represents either a straight-chain or branched-chain C₁-C₁₃-alkylene-, -alkenylene- or -alkinylene radical or the group A-B, whereby A is bonded to the estratriene skeleton and represents a benzylidene radical that is bonded via —CH₂— to the estratriene skeleton, a phenylene radical, or a C₁-C₃-alkylaryl radical that is bonded via the alkyl group to the estratriene skeleton, and B stands for a straight-chain or branched-chain C₁-C₁₃-alkylene-, -alkenylene- or -alkinylene radical, and whereby A and B can also be connected to one another via an O atom,
in which V further represents a CH₂— or a C(O) group,
in which W further is an N(R⁶)— group or an N⁺(O⁻)(R⁶) group or an azolidinylene ring or an azolidinylene-N-oxide ring, whereby the azolidinylene ring or azolidinylene-N-oxide ring includes at least one C atom of grouping X, whereby R⁶ further is either H or CH₂—R⁷ or C(O)—R⁷, in which R⁷ can mean the following:
a) hydrogen or
b) a straight-chain or branched-chain, non-fluorinated or at least partially fluorinated C₁-C₁₄-alkyl-, -alkenyl- or -alkinyl radical, which can be hydroxylated in one or more places and can be interrupted by one to three of the heteroatoms —O— and —S— and/or the groupings —NR⁹—, in which R⁹ stands for hydrogen or a C₁-C₃-alkyl radical, or
c) an unsubstituted or substituted aryl- or heteroaryl radical or
d) an unsubstituted or substituted C₃-C₁₀-cycloalkyl radical or
e) an unsubstituted or substituted C₄-C₁₅-cycloalkylalkyl radical or
f) an unsubstituted or substituted C₇-C₂₀-aralkyl radical or
g) an unsubstituted or substituted heteroaryl-C₁-C₆-alkyl radical or
h) an unsubstituted or substituted aminoalkyl radical or a biphenyl radical, in which X further is a straight-chain or branched-chain C₁-C₁₂-alkylene-, -alkenylene- or -alkinylene radical,
in which Y further is a direct bond between X and Z or can mean the following:
a) an SOₙ—R¹⁰ group, whereby n=0, 1 or 2, only if W is an N⁺(O⁻)(R⁶) group or an azolidinylene-N-oxide ring and not an N(R⁶) group or an azolidinylene ring,
whereby R¹⁰ represents a direct bond between SOₙ and Z or a straight-chain or branched-chain C₁-C₆-alkylene-, -alkenylene- or -alkinylene radical, or
b) the group R¹¹ or O—R¹¹, whereby R¹¹ stands for
i) a straight-chain or branched-chain C₁-C₅-alkylene-, -alkenylene- or -alkinylene radical or for
ii) an unsubstituted or substituted aryl radical or heteroaryl radical or for
iii) an unsubstituted or substituted C₃-C₁₀-cycloalkyl radical or for
iv) an unsubstituted or substituted C₄-C₁₅-cycloalkylalkyl radical or for
v) an unsubstituted or substituted C₇-C₂₀-aralkyl radical or for
vi) an unsubstituted or substituted heteroaryl-C₁-C₆-alkyl radical, or
c) the grouping CH═CF or
d) the grouping HN—C(O)—NH—R¹², whereby R¹² stands for an unsubstituted or substituted arylene radical, and whereby R¹² is bonded to Z, and in which Z further is a direct bond between Y and E or a straight-chain or branched-chain C₁-C₉-alkylene-, -alkenylene- or -alkinylene radical, which can be partially or completely fluorinated, and
in which E further is a CF₃ group or an at least partially fluorinated aryl group,
as well as their pharmacologically compatible acid addition salts and esters.

3. A pharmaceutical combination according to claim 1, wherein the pure antiestrogen is 11β-Fluoro-17α-methyl-7α-{5-[methyl-(8,8,9,9,9-pentafluorononyl)amino]pentyl}-estra-1,3,5(10)-triene-3,17β-diol or 7 alpha-[9-(4,4,5,5,5-pentafluoropentylsulfinyl)-nonyl]-estra-1,3,5(10)-triene-3,17 beta-diol, and their pharmaceutically acceptable derivatives or analogues thereof.

4. A pharmaceutical combination according to claim 1, wherein the pure antiestrogen is TAS-108 and ICI 164.384.

5. A pharmaceutical combination according to claim 1, wherein the weight ratio of the progesterone-receptor antagonist and the pure antiestrogen is from 1:100 to 100:1.

6. A pharmaceutical combination according to claim 1, wherein the weight ratio of the progesterone-receptor antagonist and the pure antiestrogen is from 1:4 to 4:1.

7. A pharmaceutical combination according to claim 1, wherein the progesterone-receptor antagonist is present in a unit dose of 0.1 to 100 mg and the pure antiestrogen is present in a unit dose of 0.1 to 200 mg.

8. A pharmaceutical combination according to claim 1, wherein the progesterone-receptor antagonist is present in a unit dose of 10 to 150 mg and the pure antiestrogen is present in a unit dose of 10 to 150 mg.

9. A pharmaceutically combination according to claim 1, wherein the progesterone-receptor antagonist and the pure antiestrogen is administered in the form of tablets, pills, dragees, gel capsules, granules, suppositories, implants, injectable sterile aqueous or oily solutions, suspensions, emulsions, ointments, creams, gels, patches for transdermal administration or formulations suitable for administration by inhalation.

10. A pharmaceutically combination according to claim 1, characterized that the combination comprises the progesterone-receptor antagonist 11β-(4-acetyl-phenyl)-17β-hydroxy-17α-(1,1,2,2,2-pentafluoroethyl)-estra-4,9-dien-3-one, an antioestrogen and a pharmacologically active agent.

11. A pharmaceutically combination according to claim 10, wherein the pharmacologically active agent is a cytotoxic agent.

12. A pharmaceutically combination according to claim 1 for oral administration.

13. Use of the combination according to claim 1, as medicament for the prophylaxis or treatment of BRCA1- or BRCA2-mediated breast cancer, ovarian cancer endometrial cancer, gastric cancer, colorectal cancer, endometriosis, myeloma, myoma and meningioma.

14. Use of the combination according to claim 1 for the production of a medicament for the treatment of BRCA1- or BRCA2-mediated breast cancer, ovarian cancer endometrial cancer, gastric cancer, colorectal cancer, endometriosis, myeloma, myoma and meningioma.

15. Use of the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof for the prophylaxis and treatment of BRCA1- or BRCA2-mediated breast cancer.
